(19) United States
(12) Patent Application Publication
(10) Pub. No.: US 2008/0070875 A1
(43) Pub. Date: Mar. 20, 2008

(54) Acne treatment compositions and methods of use

(76) Inventors: George P. Majewski, Redondo Beach, CA (US); Ronald J. Bohm, Colts Neck, NJ (US); Michael A. Ross, Lower Nazareth, PA (US)

Correspondence Address: DUANE MORRIS LLP;PATENT DEPARTMENT, 1540 BROADWAY, NEW YORK, NY, 10036-4086, US

(21) Appl. No.: 11/900,452

(22) Filed: Sep. 11, 2007

Related U.S. Application Data
(60) Provisional application No. 60/843,413, filed on Sep. 11, 2006.

(51) Int. Cl. A61K 31/60; A61P 17/10

(52) U.S. Cl. 514/161

(57) ABSTRACT

The present invention relates to a composition for treating acne comprising an antimicrobial/anti-inflammatory polyphenolic molecule(s) including quercetin and rosmarinic acid in combination with salicyclic acid and/or salicylate salts in a topical and/or cosmetic delivery vehicle. The invention further relates to a method for treating acne by topically administering one of the compositions in an amount therapeutically effective to reduce the redness and blemishes associated with acne.

### FIELD OF THE INVENTION

This invention relates to topical compositions and methods for treating acne by improving performance and efficacy of OTC medications including salicylic acid and salts of salicylic acid with plant derived polyphenols which provide antimicrobial and anti-inflammatory functions. The cosmetic or OTC vehicle may be any suitable form as would be recognized by one skilled in the art, including but not limited to an emulsion, gel, hydro-alcoholic or glycol vehicle which may contain other beneficial ingredients, for example, moisturizers and conditioners.

### BACKGROUND OF THE INVENTION

Acne is a skin disorder resulting from the action of hormones and other substances on the skin's oil glands (sebaceous glands) and hair follicles affects about 85% of people to some degree in their adolescent lives. In severe cases, it can cause physical and/or emotional scarring. Acne is understood to continue into adulthood for about 18% of the U.S. population.

Pilosebaceous units (PSUs) are comprised of a sebaceous (oil producing) gland connected to a hair follicle (canal). Blockage of the PSU is the major contributing factor in acne. This blockage can occur from abnormal cell build up (hyperkeratinization) on and around pores and follicles. Elevated sebum levels may lead to blocked pores thus providing an environment where the bacterium Propionbacterium acnes (P. acnes) can generate infection leading to inflammation. P. acnes is an organism that can proliferate in a clogged pilosebaceous unit once an anaerobic environment is created. Sebum also provides a food source for P. acnes. This can lead to pustule formation and an inflammatory response due to bacterial release of enzymatic and chemical agents that promote the skin's immune response. The formed pustule is known to one skilled in the art by the following terms, including but not limited to, comedo, pimple, or acne vulgaris. See “Questions and Answers about Acne” published by National Institute of Arthritis and Musculoskeletal and Skin Diseases.

The Kosho Kaishi (Journal of Perfumes and Cosmetics), Vol. 21, No. 4, page 341 (1997) states, “[t]he comedo (acne) is a chronic inflammation disease attacking folliculus pili of sebaceous gland. This is generated predominantly at the age of puberty and a large number of intrinsic and extrinsic factors are complicatedly combined to form the cause of disease. With respect to the mechanism in the crisis of comedos, importance is attached to Propionbacterium acnes (hereinafter simply referred to as P. acnes). It is considered that irritation or destruction of the folliculus pili is brought about particularly in the process of inflammation from comedo to red papula, pustule, induration or cystoma by the action of various enzymes as a product of P. acnes, such as lipase, protease and hyaluronidase, or as a result of release of lysosome enzyme from neutrophils reached folliculus pili due to a neurotaxis factor produced from P. acnes.”

Comedos have been treated using a cream or ointment containing an active drug material such as a sebum secretion inhibitor, a keratinization inhibitor (keratin abrasive), an antimicrobial or an anti-inflammatory according to the cause. However, such existing drug containing comedo treating agents are associated with negative side effects.

Mild inflammatory acne is usually treated with common OTC topical medications including benzoyl peroxide, salicylic acid or retinoids as disclosed in FDA monograph. 21 CFR 333.310 for OTC acne treatments. Benzoyl peroxide can leave the skin with an opaque or white appearance. In addition, benzoyl peroxide can bleach clothing or linens which contact the treated area. Furthermore, government regulations in some parts of the world limit or exclude the use benzoyl peroxide. For example, the European Union currently excludes the use of benzoyl peroxide in cosmetics and OTC acne treatments.

Alternatives to benzoyl peroxide are known, but are associated with problems in effectively treating patients without negative side effects. For example, formulations containing estrogen and estrogen-like drugs, such as ethinyl estradiol, have been used as sebum secretion inhibitors but these treatments are associated with nausea, headaches, weight gain, depression, thromboembolism, strokes, heart attacks and other negative side effects. Also, a commonly used keratinization inhibitor (keratin abrasive) treatment, sold under brand names such as SEBULEX, METED, PERNOX ABRADANT SCRUB CLEANSER, and SASTID, includes a sulfur compound and a salicylic acid, this combination causes excessive drying property and/or irritation. In addition, some treatments, use an antimicrobial component which includes chlorhexidine gluconate and benzalkonium chloride. These antimicrobials cause a strong irritation and extreme chapping of the skin. Furthermore antimicrobials can lead to immune strains developing in the wild-type fauna.

Comedolytic agents like salicylic acid, AHAs and the salts of both acids are popular for exfoliating dead skin cells and opening and draining the pores. Comedolytic agents have limitations and at high concentrations with very acidic pH can cause significant irritation. Comedolytic agents also have shown limited effect in preventing proliferation of P. acnes via antimicrobial activity and also have limited if any impact on directly reducing inflammation.

Benefits of compositions including quercetin with retinol and retinol derivatives for treating acne and other skin conditions including age spots and psoriasis are disclosed in U.S. Pat. No. 5,665,367 to Burger, et al. Further, treatment of rosacea also an inflammatory condition disclosed in U.S. Pat. No. 7,078,048 to Kang, et al. notes the use of quercetin as a kinase inhibitor. U.S. Pat. No. 7,074,832 to Bhagwat discloses the use of a suitable antioxidant in combination with urea for treating acne and other skin disorders.

The existing anti-inflammatory products in the marketplace have not yet succeeded in achieving satisfactory treatment of comedos. There exists a need for a treatment having a different method of action in combination that synergistically brings out the comedo treating effect. Previous attempts have failed to achieve the desired effects or have caused side effects that are problematic. Moreover, even if the above-described drugs successfully treat comedos, comedo vestiges such as pigmentation or cratered skin impression remain for a long period of time and this causes psychological problems; particularly for young people at the age of puberty. Thus, no effective combination of drugs for the comedo treatment has been attained.

There exists a need for enhancing performance and efficacy of products containing comedolytic agents including salicylic acid for topical treatment of acne. This need would be best achieved without the use of irritating, toxic or restricted agents such as benzoyl peroxide. Ideally, such an improved topical treatment addresses the problem of acne from multiple pathways, so as to provide a successful treatment for a broad population of affected people.

This invention accelerates relief from acne discomfort by allowing anti-acne compositions containing salicylic acid to offer a multi-prong activity where the exfoliation of clogged pores and follicles is now complemented with antimicrobial and anti-inflammatory activity from plant derived polyphenolic molecules including quercetin and rosmarinic acid.

### SUMMARY OF THE INVENTION

This invention offers a multi-prong approach for treating acne by using a comedolytic agent (e.g. salicylic acid) in combination with plant derived or plant identical antimicrobial/anti-inflammatory molecules including quercetin and rosmarinic acid to treat both abnormal clumping of cells in follicles while addressing by other pathways to reduce P. acnes proliferation and inflammation associated with acne.

An anti-acne composition is comprised of:

(a) 0.0001% to about 10% by weight percent of a plant derived quercetin and/or rosmarinic acid.

(b) 0.1% to about 10% by weight of salicylic acid, and alkali metal salts thereof;

(c) the remaining portion forming a delivery vehicle of cosmetic acceptance, preferably but not limited to an aqueous or hydro-alcoholic serum or emulsion base vehicle in form of water-in-oil and oil-in water and water-in-silicone.

In yet another embodiment, the quercetin and rosmarinic acid can be made soluble in a glycol based solvent including but not limited to ethoxydiglycol, propylene glycol, butylene glycol, pentylene glycol and used in that form as a topical application or added into an emulsion or gel of preference.

### BRIEF DESCRIPTION OF THE DRAWINGS

FIG. 1 depicts a chemical diagram of the accepted molecular structure of quercetin.

FIG. 2 depicts a chemical diagram of the accepted molecular structure of rosmarinic acid.

FIG. 3 depicts a chemical diagram of the accepted molecular structure of salicylic acid.

### DETAILED DESCRIPTION OF THE INVENTION

Terms

The following terms, and abbreviations are used herein and have the following definitions in this context:

The terms “acne”, “pimple”, “comedo”, “pustule” and “acne vulgaris” as used herein are synonymous and hereby used unchangeably. Such terms refer to a skin disorder that is generally characterized by hyperkeratinization on or around pores and/or follicles that is generally aggravated by the bacterium P. acnes.

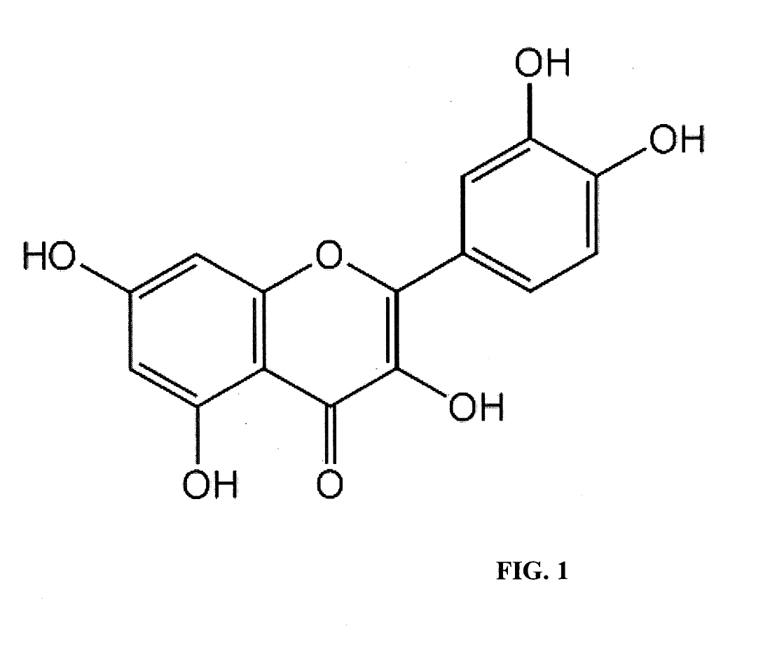

The term “quercetin” as used herein refers to a member of the family of flavonoids, more specifically type of flavonol, also more specifically type of an aglycone flavonoid. Quercetin is synonymous with the terms “2-(3,4-dihydroxyphenyl)-3,5,7-trihydroxy-4H-1-benzopyran-4-one”, “3,3′,4′,5,7-pentahydroxy flavone” and the drawing depicted in FIG. 1. These terms and the drawing are synonymous and can be used interchangeably. Quercetin is also defined chemically by these terms and FIG. 1. “Plant derived quercetin” is quercetin obtained from a plant source or sources, including, but not limited to, caper plants (Capparis sp.), lovage plants (Levisticum officinale), apple trees (Malus sp.), tea plants (Camellia sinensis) onion plants (Allium sp.), grape vines (Vitus sp.), citrus plants (Citrus sp.) cabbages (Brassica oleracea), other berry producing plants, and leafy green vegetables.

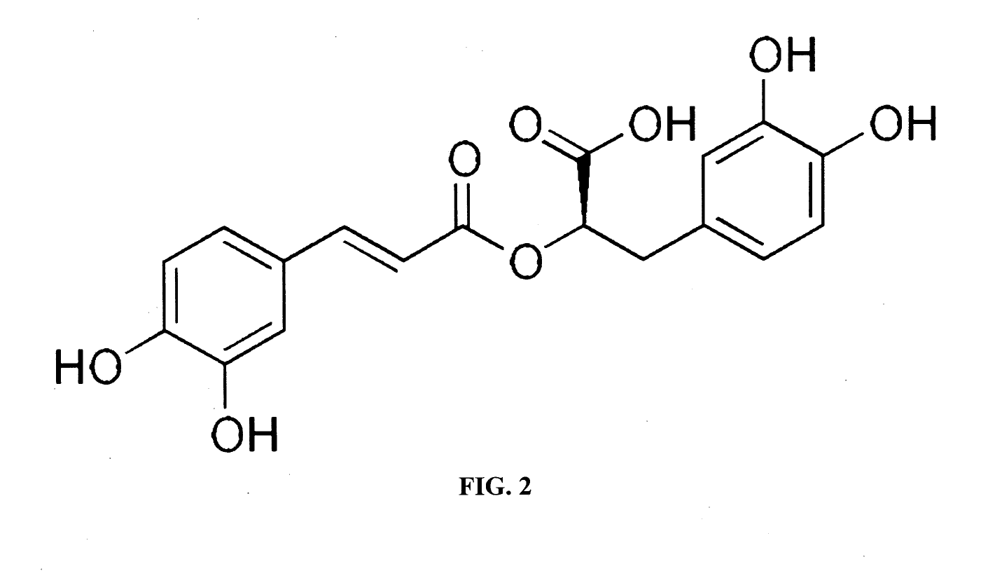

The term “rosmarinic acid” as used herein refers to a carboxylic acid known in the chemical name “(2R)-2-[[(2E)-3-(3,4-Dihydroxyphenyl)-1-oxo-2-propenyl]]oxy]-3-(3,4-dihydroxyphenyl)propanoic acid”. Rosmarinic acid can be described by the chemical structure depicted in FIG. 2. These terms and the drawing are synonymous and can be used interchangeably. As used herein, the term “rosmarinic acid” includes salts of rosmarinic acid base. Any suitable salt of rosmarinic acid can be used in accordance with the invention. Structurally, rosmarinic acid is a dimer of caffeic acid. Rosmarinic acid can be derived from plants such as rosemary (Rosmarinus officinalis), oregano (Origanum sp.), thyme (thymus sp.), sage (Salvia officinalis), peppermint (a sterile cross of Mentha aquatica and Mentha spicata) as well as other plants. In humans, rosmarinic acid is understood to be metabolized into, inter alia, methylated rosmarinic acid, coumaric acid, ferulic acid and caffeic acid.

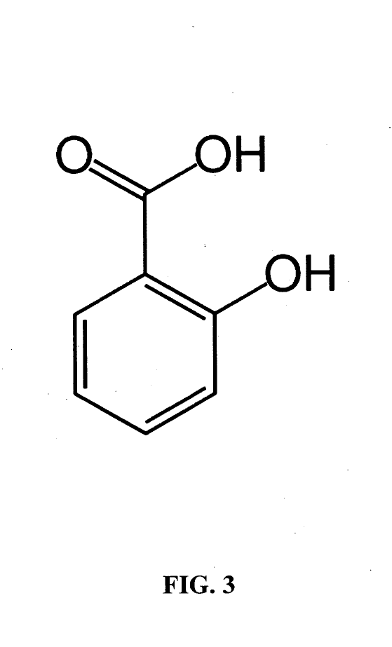

The term “salicylic acid” as used herein refers to the acid also described as 2-Hydroxybenzoic acid with the chemical formulas C₇H₆O₃ or C₆H₄(OH)CO₂H. Salicylic acid can be described by the chemical structure depicted in FIG. 3. These terms, formulas and the chemical formula are synonymous and can be used interchangeably. The term “salicylic acid”, as used herein, includes salts of salicylic acid. Any suitable salt of salicylic acid can be used in accordance with the invention.

The term “patient” as used herein refers to an animal, especially a mammal, receiving medical treatment, including over-the-counter medical treatment and preventative care. The preferred patient in the context of this invention is a human patient. The patient can be of any age or stage of development including baby, infant, toddler, preteen, teenager, and adult. The most preferred patients are in the preteen, teenager and adult stages of development. Patients can be either male or female.

The term “animal” as used here includes, but is not limited to, a human, monkey, dog, cat, horse, cow, sheep, pig, chicken, turkey, quail, mouse, rat, rabbit, and guinea pig. The animal discussed in connection with the present invention is more preferably a mammal, and most, preferably a human.

The term “plant derived” as used herein means a substance that has been isolated, extracted or produced from plant matter. Such plant matter can be cultivated in an agricultural setting, for a non-limited example, a farm, or could have grown as part of the wild or uncultivated fauna. Plant matter includes that which has been enhanced through fertilization, cross-breeding and genetic modification.

The abbreviation “QS” is used herein for the Latin term, quantum sufficiat.

The abbreviation “C” when used herein as a suffix of a number indicates the word Celsius and further indicates a temperature on the Celsius scale.

The abbreviation “mg” is used herein for the unit milligram.

The abbreviation “μg” is used herein for the unit microgram.

The abbreviation “L” is used herein for the unit liter.

The abbreviation “ml” is used herein for the unit milliliter.

The abbreviation “mol” is used herein for the unit mole, or the quantity of a substance approximately equal to 6.022×10²³ entities of said substance.

The abbreviation “CFU” is used herein for the phrase, colony forming unit.

The abbreviation “cps” is used herein for the unit centipoise.

Formulation Components

Quercetin

Quercetin is a member of the family of flavonoids. More specifically it is type of flavonol. Also more specifically it is type of an aglycone flavonoid.

Quercetin can be derived from a variety of plants. In particular quercetin can be derived from caper plants (Capparis sp.), lovage plants (Levisticum officinale), apple trees (Malus sp.), tea plants (Camellia sinensis) onion plants (Allium sp.), grape vines (Vitus sp.), citrus plants (Citrus sp.) cabbages (Brassica oleracea), other berry producing plants and leafy green vegetables.

Quercetin of any origin can be used with the present invention. This includes that which is extracted from natural sources, and quercetin that is synthesized chemically or bio-chemically in a industrial or laboratory environment. Natural origins include plants of agricultural cultivation and wild-type plants. The preferred source of quercetin is plant derived.

Quercetin demonstrates anti-inflammatory properties when applied medically to animals. Quercetin has among the highest levels of anti-inflammatory properties of the flavoid family. While not wishing to be bound by theory, it is believed that quercetin conveys its anti-inflammatory activity by inhibiting both the production of and the release of inflammation mediators such as, but not limited to, histamine.

Quercetin is further capable of acting as a potent antioxidant. Quercetin as an antioxidant protects cells against the damaging effects of reactive oxygen species, such as, but not limited to, singlet oxygen, superoxide, peroxyl radicals, hydroxyl radicals and peroxynitrite. In animals, an imbalance between antioxidants and reactive oxygen species results in oxidative stress, leading to cellular damage. Such cellular damage increases local inflammation and contributes to the conditions that promote comedo formation.

Quercetin provides both anti-inflammation and antioxidant properties to assist in preventing and clearing acne in a multi-prong approach.

Quercetin provides additional benefits beyond its anti-acne properties. Studies have linked antioxidant properties such as those demonstrated in quercetin to eliminating the oxidative stress associated with aging, atherosclerosis, cancer, ischemic injury, and neurodegenerative diseases, including but not limited to Parkinson's disease and Alzheimer's disease. Quercetin has also been shown to have anti-tumor properties.

Rosmarinic Acid

Rosmarinic acid is a natural polyphenol antioxidant carboxylic acid. It is commonly found in plant species of the Lamiaceae family or mint family; however, it may be found in other plants as well. Rosmarinic acid occurs in notably high concentration in rosemary (Rosmarinus officinalis) from whence it is given its name. The acid is also found in oregano (Origanum sp.), thyme (thymus sp.), sage (Salvia officinalis), peppermint (a sterile cross of Mentha aquatica and Mentha spicata), as well as other plants.

Any rosmarinic acid can be use in the practice of this invention. This includes rosmarinic acid that has been extracted from plants of natural sources, as well as, rosmarinic acid that has been synthesized chemically or bio-chemically in a laboratory or industrial setting. Natural origins include plants of agricultural cultivation and wild-type plants. The preferred form of rosmarinic acid for use with the present invention is plant derived.

Any salt of rosmarinic acid can be used with the present invention. Examples of the salts employable as the rosmarinic acid derivatives include, but are not limited to, salts with cations of ammonium, sodium, potassium, magnesium, calcium, strontium, barium, aluminum, iron, zinc, bismuth and organic amines. A salt with at least one of these cations is preferable. More preferable is a sodium, potassium, magnesium or zinc salt, still more preferable is a sodium, potassium or zinc salt, and particularly preferable is a sodium salt. The above-mentioned compounds in the form of water or crystal water addition products may also be used as the rosmarinic acid derivatives.

Like quercetin, rosmarinic acid is a potent antioxidant. Rosmarinic acid has greater antioxidant activity than Vitamin E which is well known as an antioxidant. Like quercetin it is believed the antioxidant properties of rosmarinic acid protects cells against the damaging effects of reactive oxygen species, such as, but not limited to, singlet oxygen, superoxide, peroxyl radicals, hydroxyl radicals and peroxynitrite. In animals, an imbalance between antioxidants and reactive oxygen species is thought to result in oxidative stress, leading to cellular damage. Such cellular damage increases local inflammation and contributes to the conditions that promote comedo formation.

Similar to quercetin, rosmarinic acid also has anti-inflammatory properties. Historically, the plant perilla, which is rich in rosmarinic acid, is used for its anti-allergic activity. A study by Sanbongi et al. (Clinical and Experimental Allergy, 34 (6): 971-977, June 2004) have shown that the oral administration of rosmarinic acid is an effective intervention for allergic asthma. Another study by Youn J. et al. (Journal of Rheumatology, 30 (6): 1203-7, June 2003) demonstrated that rosmarinic acid suppressed synovitis in mice and that it may be beneficial for the treatment of rheumatoid arthritis. Unlike antihistamines, rosmarinic acid prevents the activation of immune responder cells, which cause swelling and fluid formation.

Rosmarinic acid provides both anti-inflammation and antioxidant properties to assist in preventing and clearing acne in a multi-prong approach.

Salicylic Acid

Salicylic acid can be derived from the willow tree bark. However, the acid can also be chemically prepared from other molecules for a non-limiting example, from sodium hydroxide, phenoxide and carbon dioxide followed by acidification. Preferably, salicylic acid used in the present invention is plant derived, most preferably the acid is derived from the willow tree, species belonging to the Salicaceae family.

Salicylic acid is know to be used in cosmetic applications. Used alone salicylic acid is not as effective in the treatment of acne as other known acne treatments such as benozoic acid. Used in high concentrations, such as those approaching 100% solution, salicylic acid is damaging to human skin, and destructive to DNA. However, salicylic acid or its salt can be used safely in cosmetic applications in concentrations from about 0.1% to about 10% by weight.

Any salt of salicylic acid can be used with the present invention. Examples of the salts employable as the salicylic acid derivatives include, but are not limited to, salts with cations of ammonium, sodium, potassium, magnesium, calcium, strontium, barium, aluminum, iron, zinc, bismuth and organic amines. A salt with at least one of these cations is preferable. More preferable is a sodium, potassium, magnesium or zinc salt, still more preferable is a sodium, potassium or zinc salt, and particularly preferable is a sodium salt. The above-mentioned compounds in the form of water or crystal water addition products may also be used as the salicylic acid derivatives.

Optional Components

Optionally, the invention may be prepared with disodium rutinyl disulfate. When disodium rutinyl disulfate is used in a preparation with quercetin, the quercetin is thought to be hydrolyzed. In combination with disodium rutinyl disulfate, quercetin is more soluble and more potent. An example of such an embodiment of the invention is can be found in the gel of Example 9, as presented herein.

In addition to the above-mentioned active ingredients, salicylic acid and either or both rosmarinic acid and quercetin as essential components, other ingredients are commonly blended in a preparation, such as, but not limited to, a surfactant, an oil, an alcohol, a moisturizer, a thickener, an antiseptic, an antioxidant, a chelating agent, a pH adjuster, a perfume, a dye, an ultraviolet light absorbing/scattering agent, an amino acid, water and combinations thereof. These other ingredients can be combined with the essential ingredients of the present invention as appropriate for the desired patient end use. These ingredients can be used in amounts of about 0.0001% to about 99.8999% by weight.

Non-limiting examples of surfactants include nonionic surfactants such as lipophilic glycerin monostearate, self-emulsifying glycerin monostearate, polyglycerin monostearate, sorbitan monooleate, polyethylene glycol monostearate, polyoxyethylene sorbitan monooleate, polyoxyethylene cetyl ether, polyoxyethylated sterol, polyoxyethylated lanolin, polyoxyethylated beeswax and polyoxyethylene hydrogenated castor oil, anionic surfactants such as sodium stearyl phosphate, potassium palmitate, sodium cetylsulfate, sodium lauryl phosphate, triethanolamine palmitate, sodium polyoxyethylene lauryl phosphate and sodium N-acylglutamate, cationic surfactants such as stearyldimethylbenzylammonium chloride and stearyltrimethylammonium chloride, and amphoteric surfactants such as alkylaminoethylglycine chloride solution and lecithin.

Non-limiting examples of oils include vegetable oils and fats such as castor oil, olive oil, cacao oil, tsubaki oil, coconut oil, Japan wax, jojoba oil, grape seed oil and avocado oil, animal oils and fats such as mink oil and egg yolk oil, waxes such as beeswax, spermaceti, lanolin, carnauba wax and candelilla wax, hydrocarbons such as liquid paraffin, squalane, microcrystalline wax, ceresine wax, paraffin wax and petrolatum, natural and synthetic fatty acids such as lauric acid, myristic acid, stearic acid, oleic acid, isostearic acid and behenic acid, natural and synthetic higher alcohols such as cetanol, stearyl alcohol, hexyldecanol, octyldodecanol and lauryl alcohol, and esters such as isopropyl myristate, isopropyl palmitate, isopropyl adipate, octyldodecyl myristate, octyldodecyl oleate and cholesterol oleate.

Non-limiting examples of moisturizers include polyhydric alcohols such as glycerin, propylene glycol, 1,3-butylene glycol, sorbitol, polyglycerin, polyethylene glycol and dipropylene glycol, NMF ingredients such as amino acid and sodium lactate, and water-soluble polymer substances such as hyaluronic acid, collagen, muco-polysaccharide and chondroitin sulfate.

Non-limiting examples of thickeners include natural polymer substances such as sodium alginate, xanthan gum, aluminum silicate, quince seed extract, tragacanth gum and starch, and synthetic or semisynthetic polymer substances such as carbomer, acrylate and acrylamide copolymers, methyl cellulose, hydroxyethyl cellulose, carboxymethyl cellulose, soluble starch and cationized cellulose. The preferred thickener of the present invention is SIMULGEL EG (Seppic).

Non-limiting examples of chelating agents include disodium edetate, ethylenediaminetetraacetate, pyrophosphate, hexametaphosphate, citric acid, tartaric acid and gluconic acid. Examples of the pH conditioner include sodium hydroxide, triethanolamine, citric acid, sodium citrate, boric acid, borax and potassium hydrogenphosphate.

Non-limiting examples of ultraviolet absorbing/scattering agents which can be used in combination include p-amino acid type, hydroxybenzophenone type, benzofuran type, salicylic acid type, coumarin type and azole type organic ultraviolet absorbents, such as 2-hydroxy-4-methoxybenzophenone, octyldimethyl p-aminobenzoate and ethylhexyl p-methoxycinnamate, and this is used in an amount of from 0.001 to 10 mol/L. Furthermore, titanium dioxide, kaolin or talc may also be used in combination. Examples of amino acids include glycine, alanine, valine, leucine, isoleucine, serine, threonine, phenylalanine, tyrosine, tryptophan, cystine, cysteine, methionine, proline, hydroxyproline, aspartic acid, asparagine, glutamic acid, glutamine, histidine, lysine, and derivatives thereof.

The present invention may also be used in combination with between none and to about 10 mol/L of a whitening cosmetic material commonly used. Examples of the whitening cosmetic materials employable in combination include titanium dioxide, kojic acid, placenta extract, arbutin, SS arbutin, morusin, ellagic acid and chamomile extract.

In addition, a bactericide, a chelating agent, a plant extract ingredient and other ingredients commonly used may be added to the invention for preventing and treating skin diseases. Non-limiting examples of the other ingredients which can be added to the present invention individually or in combination include isopropylmethylphenol, sulfur, dipotassium glycyrrhizinate, resorcin, parabens, diisopropanolamine, cetostearyl alcohol, propylene glycol, isopropyl myristate, sodium hydrogensulfite, tretinoin, clindamycin, erythromycin, benzoyl peroxide, azelaic acid, TRICRONSAN (IRGASAN-DP300), glycyrrhizinic acid or a salt thereof such as a sodium or potassium salt thereof, triethanolamine, cypress extract, hinokitiol, edetate, propylene glycol, perilla extract, rosemary extract, rose extract, chamomile extract, melissa extract, sage extract, glycyrrhiza extract, jojoba extract, N-acyl-L-glutamic acid or a salt thereof such as a sodium salt thereof, cetanol, mukurossi extract, and squalane such as phytosqualane.

As additives which can be added to the agent for preventing and treating skin diseases according to the present invention, those described in the standards of cosmetic additives such as Keshohin Genryo Kijun-Gai Seibun Kikaku, Tsuiho (Standards of Ingredients other than those listed in JSCI, Supplement), issued by Yakuji Nippo Ltd. (1993) may be added for ordinary purposes. Specifically, additives that provide ultra-violet filters or sunscreen materials can be added. These additives include, but are not limited to, TiO₂ and ZnO. Other additives that can be added to the present invention include dispersed inorganics and pigments. Such dispersed inorganics and pigments include, but are not limited to, fumed silica, microfine pigments, particularly oxides and silicates, e.g. iron oxide, particularly coated iron oxides, and/or titanium dioxide, and ceramic materials such as boron nitride, or other solid components such as barium sulfate.

Furthermore, pharmaceutical ingredients described in the standards of pharmaceutical ingredients such as Japanese Pharmacopoeia, 13th rev., issued by Hirokawa Shoten (1996), Iryoyaku, Nippon Iyakuhin Shu (Medicinal Preparation, List of Japanese Drugs) (October, 1997), compiled by Nippon lyaku Joho Center, issued by Yakugyo Jiho Sha, and Ippan Yaku, Nippon Iyakuhin Shu (General Medicines, List of Japanese Drugs) (1998-99), 11th edition (issued on Nov. 10, 1997), pp. 1-1100, compiled by Nippon lyaku Joho Centor, issued by Yakugyo Jiho Sha may be added for ordinary purposes. Of the pharmaceutical ingredients, the following ingredients registered as drugs for external application are preferably used in combination. Specific examples of the ingredient include acrinol, alkylpolyaminoethylglycine, isopropanol, ethanol, benzalkonium chloride, benzethonium chloride, hydrochloric acid addition phenol, hydrogen peroxide solution, potassium permanganate, chlorhexidine gluconate, cresol soap, sodium hypochloride, sodium thiosulfate geraniol-denatured alcohol, thimerosal, phenol, BRONOPOL, povidone-iodine, formalin, mercurochrome, iodine, tincture of iodine, iodoform, resorcin, aluminum chlorohydroxyallantoinate, zinc oxide, white petrolatum, pigskin, erythromycin, oxytetracycline hydrochloride, oxytetracycline hydrochloride polymyxin B sulfate, gramicidin S hydrochloride streptomycin sulfate, tetracycline hydrochloride, dimethylchlorotetracycline hydrochloride, Glamycortisone, CHROMY-P, chloramphenicol, sulfadiazine, silver sulfadiazine, sulfisomidine, tetracycline, nadifloxacin, bacitracin fradiomycin sulfate, sodium fusidate, kanamycin sulfate, gentamycin sulfate, colistin sulfate, fradiomycin sulfate, fradiomycin sulfate, fradiomycin trypsin sulfate, polymyxin B sulfate, acrinol tincture oil, azulene, ethyl aminobenzoate, amcinonide, aluminum chlorohydroxyallantoinate, aqueous ammonia, indomethacin, ufenamate, Eksalb, isothipendyl hydrochloride, oxytetracyclinehydrocortisone hydrochloride, tetracycline hydrochloride hydrocortisone acetate, EURICH, antiphlogistic analgesic compound for external application, calamine, carbazochrom alkylpolyaminoethylglycine hydrochloride, camphor, prednisolone acetate valerate, diflucortolone valerate, dexamethasone valerate, betamethasone valerate, betamethasone valerate gentamycin sulfate, Strong Restamin Cortisone, glycyrrhezinic acid, crotamiton, ketoprofen, KENACORT A, KENACORT AG, diflorasone acetate, dexamethasone acetate, lead acetate, hydrocortisone acetate, methyl prednisolone acetate, methyl salicylate, zinc chloride, SHIUNKO, diphenhydramine, DIFLUPREDONATE, betamethasone dipropionate, bismuth subgallate, calcium hydroxide, suprofen, marronier seed extract, defatted soybean dry distilled tar, defatted soybean dry distillated tar diphenhydramine, tannic acid, dexamethasone, dexamethasone defatted soybean dry distilled tar, capsicum tincture, tocopherol vitamin A oil, triamcinolone acetonide, halcinonide, vitamin A, hydrocortisone crotamiton, FUFUMETHASONE pivalate, PYRIDORETIN, pyroxicam, phenol zinc white liniment, felbinac, PTESONIDO, bufexamac, momethasone furancarboxylic acid, fluocinonide, fluocinolone acetonide, fludroxycortide, flurbiprofen, prednisolone, alclometasone propionate, clobetasol propionate, dexamethasone propionate, deprodone propionate, BECROMETHASONE propionate, betamethasone, heparin analogs, bendazac, Mobilat, lauryl diphenhydramine sulfate, CLOBETASONE butyrate, hydrocortisone butyrate, betamethasone butyrate propionate, hydrocortisone butyrate propionate, potassium aluminum sulfate, fradiomycin ammonium sulfate betamethasone valerate, fradiomycin ammonium sulfate fluocinolone acetonide, fradiomycin ammonium sulfate prednisolone, sulfur, undecylenic acid, zinc undecylenate, undecylenic acid zinc undecylenate, undecylenic acid salicylic acid, AMOROLFIN hydrochloride, croconazole hydrochlorite, terbinafine hydrochloride, neticonazole hydrochloride, butenafine hydrochloride, clotrimazole, ketoconazole, ciclopirox olamine salicylate, siccanin, isoconazole nitrate, ECONAZOLE nitrate, oxyconazole nitrate, sulconazole nitrate, STARCH MERCURY, thioconazole, trichomycin, TRICYCLATE, tolnaftate, trinaphthate chlorhexidine hydrochloride, nystatin, variotin, BIHONAZOLE, phenyl iodoundecynoate, miconazole, wood tar, lanoconazole, sulfur camphor, potash soap, cantharis, glycerinated potash, acetic acid, salicyclic acid, silver nitrate, urea, carpronium hydrochloride, aiprostadil, diaphenylsulfone, purified saccharose povidone-iodine, TACALSITOL, tritinoin tocoferil, bucladesine sodium, heparin sodium, methoxsalen, Meladinine, ibuprofen, ibuprofen picol, young bovine blood extract and iodine. Such an ingredient or combinations thereof may be added and used in combination in an amount of from none to about 99.8999 percent by weight

Vitamins and their precursors can be added to the compositions of the present invention. Any vitamin can be added to the composition including but not limited to retinols, vitamin C and Vitamin E. Biologic organic molecules can also be added to the present invention, including but not limited to, proteins and peptides. Examples of proteins and peptides that can be added include, but are not limited to, all of the essential amino acids, collagen, keratin, elastin, and serum albumin.

Combinations

The present invention combines either or both quercetin and rosmarinic acid with salicylic acid to produce an anti-acne composition. Such composition contains from about 0.0001% to about 10% by weight of quercetin, rosmarinic acid and/or the combination of thereof with from about 0.1% to about 10% by weight of salicylic acid.

The remaining proportion of the composition can be composed of any known cosmetic or medical ingredients, in any combination or percentage. Examples of such ingredients are listed above. Such ingredients and combinations are well know to those skilled in the art. Such ingredients and combinations are not limited to those that are specifically taught or implied in the examples presented herein.

Such compositions can be prepared according to any suitable method of preparation. Such methods of preparation are well know to those skilled in the art. Such methods of preparation include but are not limited to those that are described in the examples presented herein.

The present invention may be prepared including the following specifications:

An anti-acne composition is comprised of:

(a) From about 0.0001% to about 10% by weight of a plant derived quercetin and/or rosmarinic acid.

(b) From about 0.1% to about 10% by weight of salicylic acid, and alkali metal salts thereof;

(c) the remaining portion forming a delivery vehicle of cosmetic acceptance, preferably but not limited to an aqueous or hydro-alcoholic serum or emulsion base vehicle in form of water-in-oil and oil-in water and water-in-silicone. In yet another embodiment, the Quercetin and Rosmarinic acid can be made soluble in a glycol based solvent including but not limited to ethoxydiglycol, propylene glycol, butylene glycol, pentylene glycol and used in that form as topical application or added into and emulsion or gel of preference.

More preferred embodiments of the invention provide the plant derived quercetin or rosmarinic acid or combination thereof in a concentration of between about 0.003 to about 8 percent by weight of the total composition. The most preferred embodiments of the invention provide plant derived quercetin or rosmarinic acid or combination thereof in a concentration of between about 0.1 to about 5 percent by weight of the total composition.

Other more preferred embodiments of the invention provide salicylic acid in a concentration of between about 0.2 and about 5 percent by weight of the total composition. Other most preferred embodiments of the invention provide salicylic acid in a concentration of about 0.5 percent about 2 percent by weight of the total composition.

Other optional ingredients in the serum or emulsion may include one or more of the following classes of ingredients:

(a) specialty emollients, perfumes and essential oils,

(b) vitamins and their precursors including retinols, vitamin C and Vitamin E

(c) Moisturizers and delivery agents including glycerin, lecithin and other lipids

(d) dispersed inorganics and pigments, including fumed silica, microfine pigments, particularly oxides and silicates, e.g. iron oxide, particularly coated iron oxides, and/or titanium dioxide, and ceramic materials such as boron nitride, or other solid components such as barium sulfate.

(e) proteins and peptides.

(f) Sebum regulating agents including silica, MATIPURE (LM Cosmetics) and other absorbing material as well as 5-alpha reductase inhibitors LINUMINE (LM Cosmetics)

(g) polymeric stabilizers and thickeners including xanthan gum, carbomer, acrylate and acrylamide copolymers, preferably SIMULGEL EG (Seppic) can be employed to help stabilize the emulsion.

(h) UV filters or sunscreen materials, including chemical sunscreens and dispersed physical sunscreens, including those based on titanium dioxide or zinc oxide.

The present invention can be formulated into a kit. In such a kit at least the essential ingredients of the present invention are provided in a concentrated strength such that the kit can be added during the preparation of a cosmetic formulation or to an prepared cosmetic formulation. The addition of such a kit would provide or enhance anti-acne properties of the formulation it is added to. Preferably the kit is prepared to provide quercetin or rosmarinic acid or combination thereof at a final concentration of between about 0.0001 and about 10 percent by weight of the total final composition and also provide salicylic acid at a final concentration of between about 0.1 and about 10 percent of the final composition. More preferably the kit is designed to provide a final concentration of the quercetin or rosmarinic acid or combination thereof between about 0.003 and about 8 percent of the final composition. Most preferably the kit is designed to provide a final concentration of the quercetin or rosmarinic acid or combination thereof between about 0.1 and about 5 percent of the final composition. Also more preferred is a kit which provides a final concentration of the salicylic acid of between about 0.2 and about 5 percent of the final composition. Also most preferred is a kit which provides a final concentration of the salicylic acid of between about 0.5 and about 2 percent of the final composition. Any kit of any concentration that could be diluted to an effective concentration could be used. However, the preferred kits are prepared to provide effective concentration of the essential ingredients when diluted between about 1:1 to about 1:1000.

### EXAMPLES

Those skilled in the art will be readily ascertain the broad range of use of the present invention from the examples presented. Such examples should not be considered as limiting, but and illustrative of part of the breath of use of the present invention.

### Example 1

An anti-acne cream was prepared with the following materials and methods.

| Ingredient | Percent by weight |
|---|---|
| Aristoflex AVC | 0.20% |
| Keltrol CG | 0.25% |
| Tetrasodium EDTA | 0.05% |
| Glycerin | 10.00% |
| Ethoxydiglycol | 10.00% |
| Salicylic Acid | 0.50% |
| Solublized quercetin in a | 0.50% |
| glycol-based raw material | |
| Chlorphenesin | 0.30% |
| Diocide | 1.00% |
| SIMULSOL 165 | 2.50% |
| FINSOLV TN | 5.00% |
| CRODACOL CS-50 | 1.00% |
| (Croda) | |
| Water | QS |

The above ingredients were prepared a under standard oil-in-water emulsion procedures well-known to those skilled in the art.

### Example 2

An anti-acne cream was prepared using a hydroalcoholic system using the following materials and methods.

| Ingredient | Percent by weight |
|---|---|
| Alcohol | 25.00% |
| ARISTOFLEX AVC | 1.10% |
| Glycerin | 5.00% |
| Quercetin | 5.00% |
| Water | QS |

The cream was prepared using a simple cold process dispersion of the ingredients as known by one skilled in the art.

### Example 3

An anti-acne cream was prepared with the following materials and methods.

| Ingredient | Percent by weight |
|---|---|
| ARISTOFLEX AVC | 0.20% |
| KELTROL CG | 0.25% |
| Tetrasodium EDTA | 0.05% |
| Glycerin | 10.00% |
| Ethoxydiglycol | 10.00% |
| Salicylic Acid | 2.00% |
| Solublized quercetin in a | 5.00% |
| glycol-based raw material | |
| Green Tea Polyphenols | 0.10% |
| Chlorphenesin | 0.30% |
| Diocide | 1.00% |
| SIMULSOL 165 | 2.50% |
| FINSOLV TN | 5.00% |
| CRODACOL CS-50 | 1.00% |
| Water | QS |

The cream was prepared under standard oil-in-water emulsion procedures as well know to one skilled in the art.

### Example 4

An anti-acne cream was prepared with the following materials and methods.

| Ingredient | Percent by weight |
|---|---|
| ARISTOFLEX AVC | 0.20% |
| KELTROL CG | 0.25% |
| Tetrasodium EDTA | 0.05% |
| Glycerin | 10.00% |
| Ethoxydiglycol | 10.00% |
| Salicylic Acid | 0.50% |
| Solublized quercetin in a | 0.50% |
| glycol-based raw material | |
| Rosmarinic Acid | 3.00% |
| Chlorphenesin | 0.30% |
| Diocide | 1.00% |
| SIMULSOL 165 | 2.50% |
| FINSOLV TN | 5.00% |
| CRODACOL CS-50 | 1.00% |
| Water | QS |

The cream was prepared under standard oil-in-water emulsion procedures as well known to one skilled in the art.

### Example 5

An anti-acne lotion was prepared with the following materials and methods.

| Ingredient | Percent by weight |
|---|---|
| ABIL EM90 | 2.50% |
| Mineral Oil | 15.00% |
| Octyl Stearate | 5.00% |
| GRANSIL GCM-5 | 2.50% |
| Bentone 38 | 0.50% |
| Propylene Carbonate | 0.20% |
| Mineral Oil | 2.50% |
| Sodium Chloride | 0.50% |
| GLYDANT PLUS | 0.40% |
| Ethoxydiglycol | 3.0% |
| Salicylic Acid | 1.0% |
| Quercetin | 3.0% |
| Water | QS |

The lotion was prepared under standard water-in-oil emulsion procedures as well known to one skilled in the art.

### Example 6

An anti-acne glycol serum was prepared with the following materials and methods.

| Ingredient | Percent by weight |
|---|---|
| Salicylic Acid | 1.0% |
| Quercetin | 2.0% |
| Pentalene Glycol | 20% |
| Ethoxydiglycol | QS |

The serum was prepared under standard cold dispersion procedures as well known to one skilled in the art.

### Example 7

An anhydrous acne treatment was prepared with the following materials and methods.

| Ingredient | Percent by weight |
|---|---|
| Cyclomethicone | 32.50 |
| GRANSIL PC-12 (Isododecane | 26.50 |
| and Polysilicone-11) | |
| GRANACTIVE RD-101 | 25.00 |
| (Cyclopentasiloxane, | |
| Stearoxymethicone/Dimethicone | |
| Copolymer, Dimethyl | |
| Isosorbide, Dimethicone, and | |
| Hydroxypinacolone Retinoate) | |
| NANOSPERSE ID | 5.00 |
| (Quaternium-90 Bentonite, | |
| Isododecane, and Propylene | |
| Carbonate) | |
| Salicylic Acid | 1.00 |
| Ethoxydiglycol | 3.00 |
| Natural Finish Velvet Kaolin | 2.00 |
| (Kaolin, | |
| Dimethicone/Methicone | |
| Copolymer, and Dimethicone) | |
| Anti-acne agent | 5.00 |

The anti-acne agent of this example is a mixture that was prepared in advance. The anti-acne agent contained butylene glycol, dimethyl isosorbide, and quercetin. Specifically, the anti-agent compound contained between about 45% to about 55%, by weight, butylene glycol; between about 35% to about 45%, by weight, dimethyl isosorbide; and, between about 1% to about 6%, by weight, quercetin.

The anhydrous acne treatment was prepared using the above ingredients and the following steps. The cyclomethicone and the GRANSIL PC-12 were combined in a container and mixed until uniform. Then the GRANACTIVE RD-101 and the NANOSPERSE ID were premixed together then added to the container. Afterwards, the salicylic acid and the ethoxydiglycol were premixed together and then added the container. Then the NATURAL FINISH VELVET KAOLIN was added to the container. At this stage, the ingredients in the container were mixed until uniformly dispersed. Last, the anti-acne agent was added to the container. The ingredients in the container were then mixed until uniformly dispersed.

### Example 8

An acne cleanser was prepared with the following materials and methods.

| Ingredients | Percent by weight |
|---|---|
| Water | QS |
| Propylene glycol | 2.00 |
| Bioterge AS-40 CG-P (Sodium | 26.00 |
| C14-16 olefin sulfonate) | |
| STEPAN-MILD SL3 | 2.00 |
| (Disodium Laureth | |
| Sulfosuccinate) | |
| Salicylic acid | 2.00 |
| PEG-150 Distearate | 1.50 |
| Anti-acne agent | 3.00 |
| (Ethoxydiglycol, quercetin, | |
| water, lecithin, C12-16 | |
| alcohols, and palmitic acid) | |
| GLYDANT PLUS (DMDM | 0.20 |
| Hydantoin and Iodopropynyl | |
| Butylcarbamate) | |
| Sodium hydroxide | QS |
| Sodium chloride | QS |

The anti-acne agent of this example is a mixture that was prepared in advance. The anti-acne agent contained ethoxydiglycol, quercetin, water, lecithin, C12-16 alcohols, and palmitic acid. Specifically, the anti-agent compound contained between about 85% to about 88%, by weight, ethoxydiglycol; between about 3% to about 6%, by weight, quercetin; between about 7% to about 10%, by weight, water; and, less than 1% of the following: lecithin, C12-16 alcohols, and palmitic acid.

The acne cleanser of this example was prepared using the above ingredients and the following steps. The water, propylene glycol, BIOTERGE AS-40 CG-P and the STEPAN-MILD SL3 were combined and heated to 50 C. These ingredients were agitated until the mixture was about clear in appearance. While the mixture was kept at 50 C, the salicylic acid was added. The mixture was again agitated until it was about uniform in appearance. At this point with the temperature steady at 50 C, the PEG-150 Distearate was added to the mixture. The mixture was agitated until the PEG-150 Distearate had dissolved. The mixture was then cooled. As the mixture was below 45 C but above 40 C, the anti-acne agent was added. The mixture was agitated until it was about uniform in appearance. When the mixture reached 40 C, the GLYDANT PLUS was added and agitated it was until about uniform in appearance. The mixture was cooled until it reached about room temperature. With the mixture at about room temperature the pH was adjusted to about 4.5 using a 50% sodium hydroxide solution. Next, the viscosity was adjusted using sodium chloride to 60,000 cps. Finally, additional water was added to reach the final volume.

### Example 9

A water soluble anti-acne gel was prepared with the following materials and methods.

| Ingredient | Percent by weight |
|---|---|
| Water | QS |
| Disodium EDTA | 0.20 |
| Silica | 0.50 |
| Benzophenone-4 | 0.20 |
| Hydroxyethylcellulose | 0.90 |
| Xanthan Gum | 0.20 |
| Betaine | 1.00 |
| Butylene glycol | 7.50 |
| Glycerin | 3.00 |
| Salicylic acid | 0.50 |
| Methyl dihydroxybenzoate | 0.10 |
| Anti-acne agent | 7.50 |
| Disodium rutinyl disulfate | 0.50 |
| Anti-glycagen agent | 1.00 |
| OXYNEX K (PEG-8, | 0.10 |
| tocopherol, ascorbyl palmitate, | |
| ascorbic acid, and citric acid) | |
| Diethylhexyl | 0.30 |
| Syringylidenemalonate | |
| Ethanol alcohol (denatured) | 7.50 |
| CETYL PEG/PPG-10/1 | 0.30 |
| Dimethicone | |
| Decyl Glucoside | 0.25 |
| Diocide (Caprylyl glycol, | 1.00 |
| phenoxyethanol, and hexylene | |
| glycol) | |
| Sodium Hydroxide | QS |

The anti-acne agent of this example is a mixture that was prepared in advance. The anti-acne agent contained ethoxydiglycol, quercetin, water, lecithin, C12-16 alcohols, and palmitic acid. Specifically, the anti-agent compound contained between about 85% to about 88%, by weight, ethoxydiglycol; between about 3% to about 6%, by weight, quercetin; between about 7% to about 10%, by weight, water; and, less than 1% of the following: lecithin, C12-16 alcohols, and palmitic acid.

The anti-glycagen agent of this example is a mixture that was prepared in advance. The anti-glycagen agent contained humic acids, butylene glycol, Olea europaea (Olive) fruit extract and water. Specifically, anti-glycagen agent contained between about 3% to about 10%, by weight, humic acids; less than one percent by weight of each butylene glycol and Olea europaea (Olive) fruit extract; and, water QS.

The anti-acne gel of this example was prepared using the above ingredients and the following steps. A main mixture was prepared by combining the water, disodium EDTA, silica, and benzophenone-4. These ingredients were agitated until about uniformly dispersed. The main mixture was then heated to a temperature of about 75 C to about 80 C. After the target temperature was reached, the hydroxyethylcellulose was added. A temperature range of between about 75 C to about 80 C was maintained as the mixture was agitated until the hydroxyethylcellulose was about fully hydrated. With the temperature maintained, the xanthan gum was added and the mixture was agitated until the gum appeared about uniformly dispersed in the mixture. At this point the mixture was cooled. While cooling, the betaine was added and agitated until it appeared about uniformly dispersed in the mixture.

Three side mixtures were prepared. The butylene glycol, glycerin, salicylic acid, and methyl dihydroxybenzoate were premixed in a separate container until about uniformly dispersed. These ingredients were heated to about 45 C. Meanwhile, in yet another separate container the water, humic acids, butylene glycol, and Olea europaea (Olive) fruit extract were combined and agitated in these ingredients were about uniformly dispersed. Also, in still another separate container, the OXYNEX K, diethylhexyl syringylidenemalonate, denatured ethanol alcohol, CETYL PEG/PPG-10/1 Dimethicone, and decyl glucoside were combined and agitated until the ingredients appeared about uniformly dispersed. These ingredients were heated to about 40 C.

When the main ingredients had cooled to about 45 C, the first group of side mixtures, containing the butylene glycol, glycerin, salicylic acid, and methyl dihydroxybenzoate, was added to the main mixture. The main mixture was agitated until the ingredients of the first premix were about uniformly dispersed within the main mixture. The main mixture was cooled further. When the main mixture was between about 40 C and about 45 C the anti-acne agent and the disodium rutinyl disulfate were added and the mixture was agitated until it appeared about uniform in appearance. Afterwards, while the main mixture still had a temperature range from about 40 C to about 45 C, the second side mixture of water, humic acids, butylene glycol, and Olea europaea (Olive) fruit extract were added. The main mixture was agitated until the second side mixture was about uniformly dispersed in the main mixture. The main mixture was further cooled. When the main mixture reached a temperature of about 40 C, the third side mixture of Oxynex K, diethylhexyl syringylidenemalonate, denatured ethanol alcohol, Cetyl PEG/PPG-10/1 Dimethicone, and decyl glucoside were added. The main mixture was agitated until the third side mixture was about uniformly dispersed in the main mixture. Afterwards, while the main mixture was still at about 40 C, the diocide was added and the main mixture was agitated until about uniform in appearance. Then the pH was adjusted to about 5.5. Finally, additional water was added to bring the completed mixture to a desired volume.

### Example 10

The objective of this example was to establish the minimal inhibitory concentration (MIC) of quercetin (Q), rosmarinic acid (RA), triclosan (T) and salicylic acid (SA) towards the bacterium P. acnes. Combinations of quercetin with rosmarinic acid 1:1 (Q+RA) and quercetin with salicylic acid (1:1) (Q+SA) were also tested.

The following methods were used in this example. P. acnes (MicroBiologics, ATCC #11827, batch #419697) was inoculated at 3 different dilutions and grown on blood agar plates for 3 days in anaerobic condition at 37 C. About 10 colonies were than suspended in thioglycollate broth in order to achieve the density equivalent to 0.5 McFarland standard. Test materials were dissolved in the thioglycollate broth at following concentrations:

Quercetin-1 mg/ml

Rosmarinic acid-2 mg/ml

Triclosan-3.3 mg/ml

Salicylic acid-7.5 mg/ml

The antimicrobial effect of the test samples was assayed by making serial dilutions for each material or the 1:1 combination thereof, followed by anaerobic incubation with bacterial agent in thioglycollate brothin a 96 well plate, at the initial density of 5×105 CFU/ml (0.001 McFarland standard). MIC was determined visually after 84 hours and confirmed by spectrophotometry at 405 nm with the BioRad 3550-UV microplate reader, using media with respective test materials without bacteria for background subtraction.

The results of the spectrophotometer readings can be found on the following table:

MIC against P. acnes

| | | | | | (Q + RA) | (Q + SA) |
|---|---|---|---|---|---|---|
| Samples | Q | RA | T | SA | Combo | Combo |
| MIC | 1:32 | 1:64 | 1:8 | <1:4 | 1:64 | 1:64 |
| (dilution) | | | | | | |
| MIC | 30 | 30 | 400 | >2000 | 24 | 58 |
| (μg/ml) | | | | | | |
| MIC (%) | 0.003 | 0.003 | 0.4 | 2 | 0.0024 | 0.0058 |

Quercetin and Rosmarinic Acid showed significant activity towards inhibiting P. acnes in this assay. To our best knowledge these compounds are not antibiotics, do not generate resistance and therefore are valuable anti-acne agents for cosmetic use (skin care products are used over prolonged periods of time and thus antibiotics may trigger bacterial resistance and decline of efficacy). Quercetin maintained activity in the presence of salicylic acid.

We claim:

1. A composition for the topical treatment of skin comprising:
(a) plant derived quercetin or plant derived rosmarinic acid or combination thereof that comprises between about 0.0001 to about 10 percent by weight of the total composition; and
(b) salicylic acid or alkali metal salts thereof or combination of salicylic acid and alkali metal salts that comprises between about 0.1 and about 10 percent by weight of the total composition.

2. The composition of claim 1 wherein the quercetin or rosmarinic acid or combination thereof is between about 0.003 and about 8 percent by weight of the total composition

3. The composition of claim 1 wherein the quercetin or rosmarinic acid or combination thereof is between about 0.1 and about 5 percent by weight of the total composition.

4. The composition of claim 1 wherein the salicylic acid or alkali metal salts thereof or combination of salicylic acid and alkali metal salts is between about 0.2 and about 5 percent by weight of the total composition.

5. The composition of claim 1 wherein the salicylic acid or alkali metal salts thereof or combination of salicylic acid and alkali metal salts is between about 0.5 and about 2 percent by weight of the total composition

6. The composition of claim 1 wherein the composition further comprises more than about 20% alcohol by weight.

7. The composition of claim 1 wherein the composition further comprises a delivery vehicle comprising an oil.

8. The composition of claim 1 wherein the composition further comprises a delivery vehicle comprising glycol.

9. The composition of claim 8 wherein the glycol is one or more glycols selected from the group consisting of ethoxidiglycol, hexylene glycol, propylene glycol, butylene glycol, and pentylene glycol.

10. The composition of claim 1 wherein the composition further comprises disodium rutinyl disulfate.

11. The composition of claim 1 wherein the skin the treatment is designed for is human skin.

12. A kit for making a topical treatment for skin comprising a solution containing:
(a) plant derived quercetin or plant derived rosmarinic acid or combination thereof that comprises between about 0.0001 to about 10 percent by weight of the total composition; and
(b) salicylic acid or alkali metal salts thereof or combination of salicylic acid and alkali metal salts that comprises between about 0.1 and about 90 percent by weight of the total composition.

13. The kit of claim 12 wherein the solution is designed to be diluted to between about 1:1 and about 1:1000 for end use.

14. The method of treating skin by applying a topical treatment comprising
(a) plant derived quercetin or plant derived rosmarinic acid or combination thereof that comprises between about 0.0001 to about 10 percent by weight of the total composition; and
(b) salicylic acid or alkali metal salts thereof or combination of salicylic acid and alkali metal salts that comprises between about 0.1 and about 10 percent by weight of the total composition.

15. The method of claim 14 wherein the quercetin or rosmarinic acid or combination thereof is between about 0.003 and about 8 percent by weight of the total composition

16. The method of claim 14 wherein the quercetin or rosmarinic acid or combination thereof is between about 0.1 and about 5 percent by weight of the total composition.

17. The method of claim 14 wherein the salicylic acid or alkali metal salts thereof or combination of salicylic acid and alkali metal salts is between about 0.2 and about 5 percent by weight of the total composition.

18. The method of claim 14 wherein the salicylic acid or alkali metal salts thereof or combination of salicylic acid and alkali metal salts is between about 0.5 and about 2 percent by weight of the total composition

19. The method of claim 14 wherein the treated skin is human skin.
